(19) United States
(12) Patent Application Publication
(10) Pub. No.: US 2019/0350537 A1
(43) Pub. Date: Nov. 21, 2019

(54) Systems And Methods For Provide For Blood Glucose Biofeedback Training And Sensory Augmentation

(71) Applicants: Matthew Lumpkin, Pasadena, CA (US); Eyad Alrabbat, Garland, TX (US); Mabelle Pasmooij, Los Altos, CA (US)

(72) Inventors: Matthew Lumpkin, Pasadena, CA (US); Eyad Alrabbat, Garland, TX (US); Mabelle Pasmooij, Los Altos, CA (US)

(21) Appl. No.: 16/417,593

(22) Filed: May 20, 2019

Related U.S. Application Data
(60) Provisional application No. 62/673,867, filed on May 19, 2018.

(51) Int. Cl. A61B 5/00; A61B 5/145

(52) U.S. Cl. CPC A61B 5/7455; A61B 5/746; A61B 5/14532

(57) ABSTRACT

The present invention relates to systems, methods and devices useful in monitoring blood glucose. Specifically, embodiments of the present invention relate to systems, methods and apparatuses that provide for biofeedback training and sensory augmentation via novel alerts.

### CROSS REFERENCE TO RELATED APPLICATIONS

The present application claims the benefit of U.S. Provisional 62/673,867, filed May 19, 2018, the entirety of which is incorporated by reference.

### TECHNICAL FIELD

The present invention relates to systems, methods and devices useful in monitoring blood glucose. Specifically, embodiments of the present invention relate to systems, methods and apparatuses that provide for biofeedback training and sensory augmentation via novel alerts.

### BACKGROUND OF THE INVENTION

Continuous glucose monitors (CGM's) are wearable sensors that measure glucose in interstitial tissue as a proxy for blood glucose. They are most commonly used by people living with type 1 diabetes but are also used by people with type 2 diabetes and by some athletes to enhance their performance through blood glucose awareness. Many such devices provide a regular output of blood glucose level to a smartphone app and can then pass that data to other apps on the phone triggering alerts, and alarms notifying the user of blood glucose level, direction and speed of change.

However, these threshold based, punctiliar alarms are problematic in numerous ways. Users report that the alarms frequently are too late to report the blood glucose and to intervene in time to prevent uncomfortable and dangerous bodily symptoms. One solution users have come up with is to set alarms thresholds to be even more sensitive and happen earlier before highs and lows. However the constant fluctuation and speed of change mean this alternative results in too many false alarms, repeating alarms, and alarm fatigue.

Furthermore, there are problems with the traditional audio and visual alarms built into these device systems and their phone app components. First, the smartphone while convenient and almost always close at hand, is already an extremely “noisy channel” in terms of alarms, alerts and bids for the user's attention. When you mix potentially lifesaving alerts and alarms into a channel that is saturated with much lower importance alerts and alarms, the efficacy of the higher priority alarms fades. Second, there is a social dimension to using loud auditory alarms designed to wake a sleeping patient from a low bg event when those alarms go off in social settings such as school, work or in public. Many patients report feeling “outed” by these alarms to friends and peers which serves to further erode their sense of agency over how and when they communicate about their disease. In addition the cognitive load of remembering to check or managing these alerts is an unnecessary additional burden imposed by this valuable technology.

Although specific advantages have been enumerated above, various embodiments may include some, none, or all of the enumerated advantages. Additionally, other technical advantages may become readily apparent to one of ordinary skill in the art after review of the following figures and description.

### BRIEF SUMMARY OF THE INVENTION

The present disclosure relates to a method and system for maintaining blood glucose level awareness in an ongoing and persistent way. It is the object of this invention to create a system comprising both specialized hardware and software that enables a number of different uses focused on providing improved, more discreet, and agency preserving threshold alarms. It is a further objective of the present disclosure to provide ongoing ambient awareness of one's blood glucose level.

Particularly, the present disclosure provides a tactile sensory cue to the wearer of a mobile device, that also provides notification of changes in their blood glucose or the blood glucose of a child for whom they are responsible. In one embodiment, the present disclosure discloses consuming the datastream output of a continuous glucose monitor and then rendering the blood glucose state and any changes in that state by means of tactile (vibratory or haptic) sensory input (also referred to as a tactile alert).

In one embodiment, the system further comprises a Biofeedback trainer which is a biofeedback cue for the user that can serve to train and enhance their innate interoception of their blood glucose level and secondary sensory cues caused by it (fatigue, light-headedness, nausea, disorientation, hunger, frustration, emotional volatility etc.).

In another embodiment, the system further comprises a Blood Glucose Sensory Augmentation which is a cue that enables the data stream from the CGM to serve as a sensory augmentation by rerouting that stream into a sensory signal through tactile input that is then picked up and integrated by the brain as a new sense that corresponds to blood glucose state.

In yet another embodiment, the system further comprises a Discreet Personal Notification of blood glucose change preserving user and or caregiver agency over if, when and how they reveal they are thinking about and managing their blood glucose levels. An additional dimension of this embodiment is to provide CGM notifications at range for users engaged in various sports and other athletic activities during which having a phone upon one's person is impossible or burdensome.

Although specific advantages have been enumerated above, various embodiments may include some, none, or all of the enumerated advantages. Additionally, other technical advantages may become readily apparent to one of ordinary skill in the art after review of the following figures and description.

### BRIEF SUMMARY OF THE DRAWINGS

FIG. 1 illustrates the overall form of a wearable device.

FIG. 2 illustrates directionality alerts related to various signals.

FIG. 3 illustrates a two part signal.

FIG. 4 describes a process for directional blood glucose biofeedback.

### DETAILED DESCRIPTION OF THE INVENTION

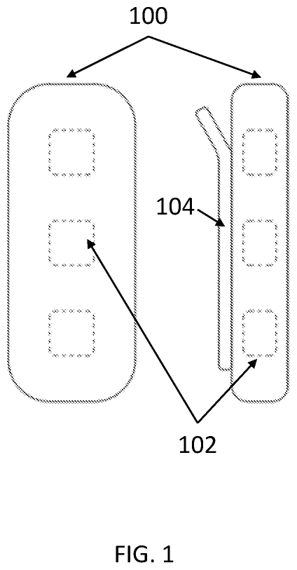

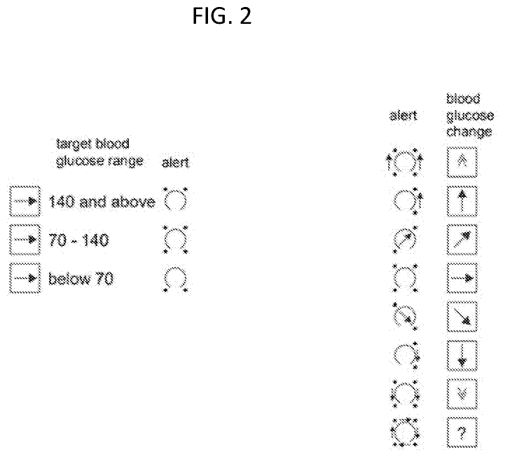

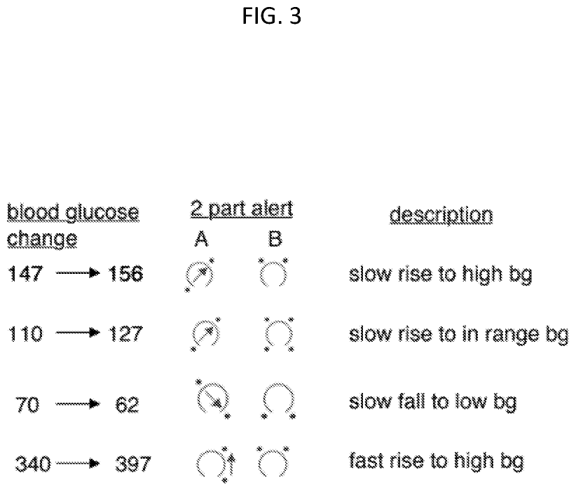

FIG. 1 illustrates embodiments of a wearable apparatus 100 according to an embodiment of the present disclosure. Embodiments of the present disclosure provide hardware designed to be used in conjunction with a smartphone and software running on the smartphone to configure settings. It can be appreciated that the hardware and software capabilities may be utilized in the following ways as described below. FIG. 1 illustrates the overall form of a wearable device with an array of two or more vibratory motors or haptic actuators 102 organized in a line, to enable directionality in data stream and or meaning transmission of both intensity and direction of blood glucose change. FIG. 2 illustrates directionality alerts related to various signals. FIG. 3 illustrates a two part signal.

In one embodiment, the present disclosure comprises a continuous glucose monitor (CGM) is transmitting a regular stream of measurements of the user's (or caregiver's child's) blood glucose level that is then transmitted via a wireless communication link to a nearby smartphone. The wireless communication link may include one or more of an RF communication protocol, an infrared communication protocol, a Bluetooth enabled communication protocol, an 802.11x wireless communication protocol, or an equivalent wireless communication protocol which provides secure, wireless communication of several units (for example, per HIPPA requirements) while avoiding potential data collision and interference. In one embodiment, the CGM is transmitting measurements every five minutes.

The smartphone (or similar device) is configured to receive the CGM's transmissions and then to share them with another app designed to run the wearable via personal health data sharing intermediaries such as Apple Health on iOS and Google Fit on Android, via CGM supplied API or directly from app to app via shared software development kits (SDK's) or other open source protocols.

Embodiments of the present disclosure further comprise an application to consume those reading from the CGM and translate them into appropriate control signals for the wearable and to provide an interface for fine-tuning the system for a given mode or functionality.

FIG. 1 illustrates a battery powered, small, discreet, wearable electronic device 100 that receives control signals wirelessly from the smartphone. This device 100 may consist of a battery (rechargeable or disposable), a control circuit board containing the relevant communications radios to enable two way communication at range, and microcontrollers to control and drive the vibratory motors and or haptic actuators 102. In one embodiment, the motors or actuators could be configured in line arrays of two or more in order to convey directionality in the datastream being transmitted.

Embodiments of the present disclosure can be used in different embodiments.

Blood Glucose Biofeedback Trainer:

In this embodiment, the process functions as a biofeedback cue for the user that can serve to train and enhance the user's innate interoception (perception of internal states) of their blood glucose level and secondary sensory cues caused by it (fatigue, light-headedness, nausea, disorientation, hunger, frustration, emotional volatility etc.)

After configuration of the system wherein data from the CGM is flowing through the system and driving the wearable, the user would then be guided by the app in a series of exercises, some planned, and some ad hoc driven by the opportunistic occurance of blood glucose states wherein trends and movement were clear that the user was moving high or low, but prior to the high or low state being high or low enough to trigger more overt secondary symptoms. This mode could provide a continuous, low level sensory cue corresponding to the CGM data, or it might also provide intermittent input that changed over time in order to provide the user with a cue. That cue might then remind them to conduct an app-guided, mindfulness-based interoceptive self-inventory of signs or symptoms that they could learn to consciously perceive without the benefit of the system with the goal of self-sensing and self-alerting earlier to uncomfortable, inconvenient and potentially dangerous highs and lows enabling earlier intervention.

Blood Glucose Sensory Augmentation

In this embodiment, the invention functions as sensory cue that enables the data stream from the CGM to serve as a sensory augmentation by rerouting the CGM data stream into a sensory signal through tactile input that is then picked up and integrated by the brain as a new sensory experience that corresponds to blood glucose state (Novich, 2014).

This is done by the stimulation of A-delta fibers, light touch sensing fibers on the surface of the skin. Once they receive a signal, they synapse to ascending tracts in the spinal cord, which then synapse in various regions of the brain, including the somatosensory cortex. Though the user may consciously feel the vibrations initially, research has shown that they will unconsciously pair the stimulus with the sense that we are seeking to acquire, in this case interoception of glucose levels. Furthermore, they can suppress the tactile stimuli through selective attention, so that they are not constantly distracted by the tactile stimuli throughout their day.

The design of the tactile stimuli and is a critical part of this invention. The stimulus must be sufficient to convey both directionality, velocity or rate of change and still communicate when bg has crossed certain critical high and low thresholds.

The stimulus may be constant low level or merely intermittent in accordance with the datastream input from the CGM (every five minutes sample rate with Dexcom's CGM). One design for example, might provide a short, flat haptic actuation to let the user know that the bg is in range and has not changed more than 5 points in either direction since the last measure. However in the event is has changed more than 5 points the vibration would be more intense and carry a directional cue corresponding to the direction of the change. This would allow the flat, “no attention needed” cue to recede in the user's attention and minimize disruption of attention to regular activity.

The further the bg goes out of range the more intense the haptic cue until it reaches an alarm state designed to wake a sleeping person (ether caregiver or patient). The objective is to create an ongoing sensory stream that when paid attention to can allow the user to maintain an ambient glucose awareness and thereby intervene early enough to avoid uncomfortable, dangerous and damaging highs and lows. But to do so in such a way as to take advantage of the brain's natural ability to integrate novel, consistent, meaningful sensory inputs into new sensory awareness.

### Discreet Personal Blood Glucose Notification:

In this embodiment, the invention functions as a discreet, personal notification of blood glucose change preserving user and or caregiver agency over if, when and how they reveal they are thinking about and managing their blood glucose levels. An additional dimension of this application is to provide CGM notifications at range for users engaged in various sports and other athletic activities during which having a phone upon one's person is impossible or burdensome.

This embodiment is perhaps the most simple in that the thresholds for notifications, alerts and alarms based on passing certain blood glucose thresholds, fall or rise rates etc. could be configured and customized via the mobile phone app. Within the app environment there would be saved configuration states for example, high intensity vibratory sensory input to get one's attention during athletic or other highly physical activities, and more subtle signals for use during a job interview or group test-taking scenario.

In the design of haptic cues translating CGM blood glucose states to the wearer of the invention there are three critical dimensions to transmit for all use cases:

Relative position to target range: Target range is usually 70-140 mg/dL (miligrams of glucose per deciliter of blood), thus a wearer may be below, within, or above their target range.

Direction of change since last reading: for every interval of reading there is either a change up or down from the last reading. So if one is at 130 mg/dL and then at the next read is at 155, their direction is up from the last read. And they have passed from within target range to above target range.

Rate of change or velocity: The rate of change in blood glucose up or down. To use the example above the wearer moved up by 15 points between reads. Let's say their next read is 20 points higher, then their velocity is high and accelerating.

Embodiments of the present disclosure provide alerts to capture these three dimensions in a number of ways. On example might be to use an array of four haptic actuators arranged around a wristband such that relative position could be encoded by whether the alert originated from the top or bottom of the wrist, the direction of change could be encoded in the movement of the vibration from bottom or top or top to bottom and the rate of change could be encoded in intensity or duration of vibration.

FIG. 2 illustrates various directionality of alerts related to various signals on a four-actuator, wrist-worn wearable device embodiment. On the left side of the figure demonstrates how the position of the latest blood glucose reading with reference to target range (70-140 mg/DL) could be translated into a haptic alert wherein the bottom two actuators could alert to convey a reading below target range, the top two could alert to convey a reading above the target range and all four to convey a reading within the target range. On the right side of the illustration, we show how an additional alert could be added to that first alert to convey direction and velocity of change in bg. In one embodiment, synchronized rising alerts on both sides of the wearable increase a significant increase in blood glucose and synchronized dropping alerts on both sides of the wearable indicate a significant decrease in blood glucose. Synchronized rising alerts on one side of the wearable increase a moderate increase in blood glucose, and synchronized dropping alerts on one side of the wearable indicate a significant decrease in blood glucose. A diagonal rising alert signifies a small increase in blood glucose, and a diagonal dropping alert signifies a small decrease in blood glucose. An alert moving around the four actuators is used to indicate an error or unreadable glucose state.

FIG. 3 illustrates other examples of a two part haptic alert and how it could be used in an embodiment with a four actuator, wrist-worn wearable device. On the left we see blood glucose change between the most recent two readings. In the Alert column A we see how a movement of haptic actuation or tactile vibration could be used to convey a slow rise, a slow fall, a fast rise or a fast fall by translating the change in blood glucose amount into a directional vibration across the wearable and the wearer's arm. The alert column B we see how the actuators are could be pulsed to show whether the current reading being conveyed is either above target range (high), in range or below range (low) such that in combination this two part actuation could make the wearer aware consciously or by means of the other methods described of current changes in blood glucose and thus enable intervention in a less disruptive and lower cognitive load way.

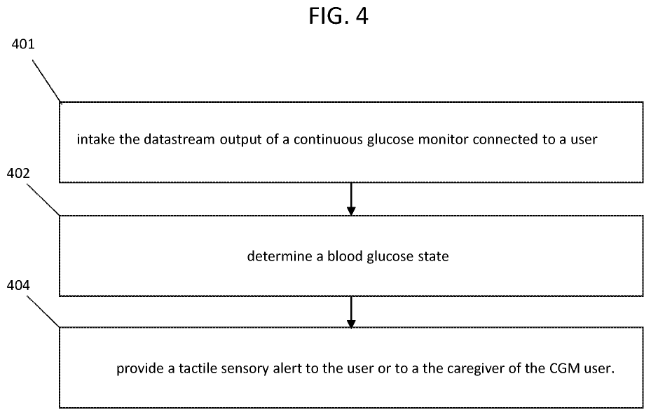

FIG. 4 illustrates a method. In step 401, the method intakes a datastream output of a continuous glucose monitor connected to a user. In step 402, the method determines a blood glucose state. In step 404, the method provides a tactile sensory alert to the user or to a caregiver of the CGM user.

Another design might encode all three dimensions into a series of alerts from a single haptic actuator capable of encoding waveforms of vibrations from, for example, 0-144 intensity level gradations. In such an embodiment relative position might be encoded in intensity of an initial haptic cue to get one's attention. In range would be minimal as it requires minimal attention. The further the wearer's excursions from that range went the greater intensity the initial cue would transmit. A second cue in sequence after the relative position could encode direction of change. A rising blood glucose cue would start in low intensity and rise in intensity. A falling blood glucose cue would start in high intensity and fall in intensity. The rate of change could be encoded in the speed or duration of the rise or fall. For very fast falls or rises a quick repetition of the second cue might be required to increase that cue's intrusion into the conscious attention of the wearer.

Biofeedback mode: more specific description of the biofeedback mode with wireframe of app guided interoception training.

The Biofeedback trainer mode works by calling the wearer's attention to their changing blood glucose state in a discreet way via haptic cue and then provides an additional guided biofeedback session, possibly in the form of a mobile app. This biofeedback session offers contextual guidance based upon blood glucose state to call the wearer's attention to secondary bodily or cognitive sensations that correlate to high or low glucose states in order to reinforce their ability to perceive, notice, attend to and ultimately consciously become aware of dangerous high and low blood glucose states without the CGM or haptic cue. In type 1 diabetes, one complication is decrease in awareness of dangerous hypoglycemic (low blood glucose) events. The longer one has the disease the less sensitive one becomes to these states. And some people with type 1 begin their life with the disease without the ability to sense their lows through interoception. This puts them at higher risk for these dangerous and life-threatening lows.

For example, imagine a wearer receives a haptic cue from a wearable on their wrist that corresponds to a high and rising blood glucose: for example, 200 mg/dL moving up at a velocity of 20 points per 5 minute interval. User's phone app would send a notification that there is a window of opportunity for biofeedback training. Upon opening the notification they would be presented with a screen that guides them in a moment of attending to their own interoceptive perception—their own sense of their own internal bodily sensations. The guided experience might log or keep track of which symptoms they most commonly associate with which blood glucose states and after a period of tracking and self-reported monitoring the experience might offer begin to pro-actively ask they are experiencing certain symptoms upon reaching previously mapped CGM supplied blood glucose states.

The present invention is not limited to the embodiments described above herein, which may be amended or modified without departing from the scope of the present invention as set forth in the appended claims, and structural and functional equivalents thereof. The Background section is incorporated by reference into the detailed description as disclosing alternative embodiments.

In methods that may be performed according to embodiments herein and that may have been described above and/or claimed below, the operations have been described in selected typographical sequences. However, the sequences have been selected and so ordered for typographical convenience and are not intended to imply any particular order for performing the operations.

1. A method, comprising:
intake a datastream output of a continuous glucose monitor connected to a user;
determine a blood glucose state based on the datastream output;
provide a tactile sensory alert to the user or to a caregiver of the CGM user.

2. The method of claim 1, wherein the tactile sensory alert is provided via a plurality of haptic actuators.

3. The method of claim 1, wherein the tactile sensory alert is provided via a single haptic actuator.

4. The method of claim 1, wherein the tactile sensory alert is provided by vibrating motors.

5. The method of claim 1, wherein the tactile sensory alert is provided by rotating mass motors.

6. The method of claim 2, wherein the plurality of haptic actuators are arranged vertically and provide directionality based blood glucose alerts.

7. The method of claim 6, wherein the directionality based blood glucose alerts further comprise alerts conveying both intensity and direction of blood glucose change.

8. The method of claim 6, wherein the sensory alert further comprises a biofeedback trainer, wherein the biofeedback trainer provides a biofeedback cue for the user that can serve to train and enhance their innate interoception of their blood glucose level.

9. The method of claim 6, wherein the sensory alert further comprises blood glucose sensory augmentation by rerouting the datastream into a sensory signal through tactile input, wherein the user's brain picks up the tactile input which is integrated by the brain as a new sense that corresponds to blood glucose state.

10. The method of claim 6 wherein synchronized rising sensory alerts on one side of the wearable increase a moderate increase in blood glucose, and wherein synchronized dropping alerts on one side of the wearable indicate a significant decrease in blood glucose, and wherein a diagonal rising alert signifies a small increase in blood glucose, and wherein a diagonal dropping alert signifies a small decrease in blood glucose
